(19) United States
(12) Patent Application Publication
(10) Pub. No.: US 2024/0172942 A1
(43) Pub. Date: May 30, 2024

(54) SPECTRUM ANALYZING METHOD AND GINGIVITIS EVALUATING DEVICE

(71) Applicant: Metal Industries Research & Development Centre, Kaohsiung (TW)

(72) Inventors: Sheng-Hung Yang, Kaohsiung City (TW); Po-Chi Hu, Kaohsiung City (TW); Yuan-Hsun Tsai, Kaohsiung City (TW); I-Wen Huang, Kaohsiung City (TW)

(73) Assignee: Metal Industries Research & Development Centre, Kaohsiung (TW)

(21) Appl. No.: 18/516,964

(22) Filed: Nov. 22, 2023

(30) Foreign Application Priority Data
Nov. 28, 2022 (TW) 111145442

(51) Int. Cl. A61B 5/00; G01J 3/28; G01J 3/42; G01J 3/457; G16H 10/60

(52) U.S. Cl. CPC A61B 5/0088; G01J 3/28; G01J 3/42; G01J 3/457; G16H 10/60; G01J 2003/2863; G01J 2003/425

(57) ABSTRACT

A spectrum analyzing method and a gingivitis evaluating device are provided. The spectrum analyzing method includes steps as follows. A diffuse reflection signal of a gingiva is calculated, and a gingiva spectrum is generated. The gingiva spectrum and a plurality of reference gingiva spectra are respectively applied with a time-series similarity calculation, and a plurality of similarity values are generated. The plurality of reference gingiva spectra correspond to various gingival indexes (GI). A minimum similarity value of the plurality of similarity values is obtained. A GI result is output according to the minimum similarity value.

### CROSS-REFERENCE TO RELATED APPLICATION

This application claims the priority benefit of Taiwan application serial no. 111145442, filed on Nov. 28, 2022. The entirety of the above-mentioned patent application is hereby incorporated by reference herein and made a part of this specification.

### BACKGROUND

### Technical Field

The disclosure relates to an evaluating method, and in particular to a spectrum analyzing method and a gingivitis evaluating device.

### Description of Related Art

Generally speaking, a doctor may use a periodontal probe to insert into the gap between the tooth and the gingiva, and determine the health status of the gingiva based on the depth of insertion of the periodontal probe. However, the current gingivitis evaluating method has to contact the tooth and gingiva, causing discomfort to the patient, and the depth of the periodontal probe has to be visually inspected, which increases the risk of misdiagnosis.

In order to solve the deficiencies caused by the contact evaluation, the industry and scholars have proposed a non-contact evaluating method. The evaluating method can analyze physiological information shown by a diffuse reflection spectrum of the gingiva at a specific wavelength to provide a basis for the diagnosis of the doctor. However, the evaluating method still relies on the understanding and judgment of the doctor with respect to the information to obtain a diagnosis result. Therefore, the current non-contact evaluating method cannot complete the diagnosis of the gingivitis efficiently or accurately.

### SUMMARY

The embodiment of the disclosure provides a spectrum analyzing method that can analyze the diffuse reflection signal of the gingiva and generate a gingivitis diagnosis result accordingly, so as to improve the efficiency and accuracy of the evaluation.

The spectrum analyzing method of the embodiment of the disclosure includes the following steps. A diffuse reflection signal of a gingiva is calculated, and a gingiva spectrum is generated. The gingiva spectrum and a plurality of reference gingiva spectra are respectively applied with a time-series similarity calculation, and a plurality of similarity values are generated. The plurality of reference gingiva spectra correspond to various gingival indexes (GI). A minimum similarity value of the plurality of similarity values is obtained. A GI result is output according to the minimum similarity value.

The embodiment of the disclosure also provides a gingivitis evaluating device. The gingivitis evaluating devices include a sensor, a spectrophotometer, and an electronic device. The sensor is configured to sense a diffuse reflection signal of a gingiva. The spectrophotometer is coupled to the sensor. The spectrophotometer is configured to calculate a diffuse reflection signal, and a gingiva spectrum is generated. The electronic device is coupled to spectrophotometer. The electronic device is configured to apply the gingiva spectrum and a plurality of reference gingiva spectra respectively with a time-series similarity calculation, and a plurality of similarity values are generated. The plurality of reference gingiva spectra correspond to various gingival indexes (GI). The electronic device is configured to obtain a minimum similarity value of the plurality of similarity values. The electronic device is configured to output a gingival index result according to the minimum similarity value.

Based on the above, in the spectrum analyzing method and the gingivitis evaluating device according to the embodiment of the disclosure, multiple similarity values may be generated based on the time-series similarity calculation and according to the gingiva spectrum to analyze the degree of similarity between the gingiva spectrum and different reference gingiva spectra. The spectrum analyzing method can generate the reference gingiva spectrum that is most similar to the gingiva spectrum according to the minimum similarity value, and the gingival index of the reference gingiva spectrum is output as the gingival index result, thereby improving the efficiency and accuracy of the evaluation.

In order to make the above-mentioned features and advantages of the disclosure more comprehensible, the embodiments are described in detail below with the accompanying drawings.

### BRIEF DESCRIPTION OF THE DRAWINGS

FIG. 1 is a schematic block diagram of a gingivitis evaluating device according to an embodiment of the disclosure.

FIG. 2 is a flow chart of a spectrum analyzing method according to an embodiment of the disclosure.

FIG. 3A to FIG. 3B are schematic operation diagrams of the gingivitis evaluating device shown in the embodiment of FIG. 1 according to the disclosure.

FIG. 4 is a schematic operation diagram of the gingivitis evaluating device shown in the embodiment of FIG. 1 according to the disclosure.

FIG. 5 is a schematic operation diagram of the gingivitis evaluating device shown in the embodiment of FIG. 1 according to the disclosure.

### DESCRIPTION OF THE EMBODIMENTS

Part of the embodiment of the disclosure will be described in detail with reference to the accompanying drawings. The reference numerals cited in the following description will be regarded as the same or similar elements when the same reference numerals appear in different drawings. The embodiments are merely part of the disclosure and do not disclose all possible implementations of the disclosure. Rather, the embodiments are merely examples within the scope of the patent application according to the disclosure.

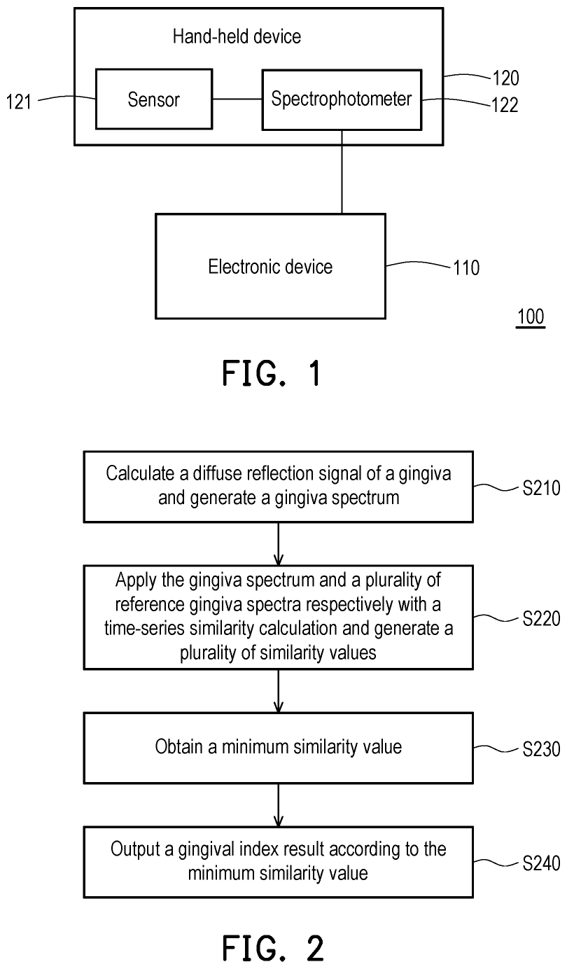

FIG. 1 is a schematic block diagram of a gingivitis evaluating device according to an embodiment of the disclosure. Referring to FIG. 1, a gingivitis evaluating device 100 may perform a non-contact evaluation on a gingiva. In this embodiment, the gingivitis evaluating device 100 may include an electronic device 110 and a hand-held device 120.

In this embodiment, the hand-held device 120 is coupled to the electronic device 110. The hand-held device 120 may collect corresponding gingiva sensing data at a tooth position to be evaluated. In this embodiment, the hand-held device 120 may include a sensor 121 and a spectrophotometer 122.

In this embodiment, the sensor 121 may include a light source (not shown). The sensor 121 is disposed in a probe of the hand-held device 120 together with the light source. The sensor 121 may sense a diffuse reflection signal generated by the gingiva with respect to the light source. In this embodiment, the sensor 121 may be, for example, an optical sensor.

In this embodiment, the spectrophotometer 122 is coupled to the sensor 121. The spectrophotometer 122 may receive the diffuse reflection signal sensed by the sensor 121. The spectrophotometer 122 may perform a spectrum analysis on the diffuse reflection signal to generate spectrum information (i.e., a gingiva spectrum).

In this embodiment, the electronic device 110 is coupled to the spectrophotometer 122. The electronic device 110 may receive the gingiva spectrum generated by the spectrophotometer 122. The electronic device 110 may diagnose a health status of the gingiva according to the gingiva spectrum (i.e., a gingival index result). In this embodiment, the electronic device 110 may be, for example, a mobile phone, a tablet computer, a laptop computer, and a desktop computer. The electronic device 110 may load and execute a computer program-related firmware or software to implement calculation and analysis functions.

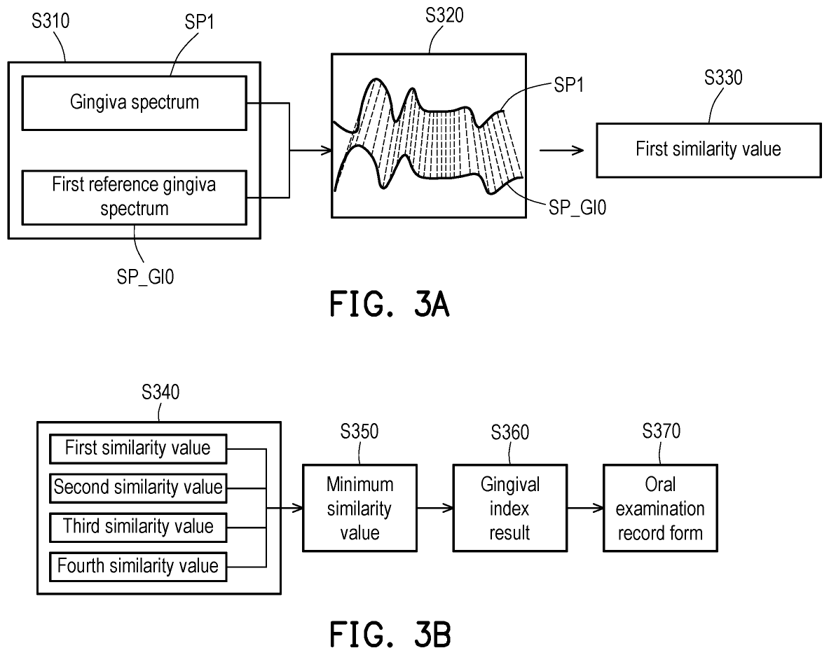

FIG. 2 is a flow chart of a spectrum analyzing method according to an embodiment of the disclosure. Referring to FIG. 1 and FIG. 2, the gingivitis evaluating device 100 may execute the following Steps S210 to S240 to evaluate the health status of the gingiva. In this embodiment, Steps S210 to S240 may be applied to the following exemplary situations.

In this embodiment, the light source of sensor 121 emits a pulse signal to the gingiva to be evaluated. The sensor 121 senses the diffuse reflection signal of the gingiva and transmits the sensed diffuse reflection signal to the spectrophotometer 122. In this embodiment, the wavelength of the pulse signal may be, for example, in a range of 400 nm to 800 nm.

In Step S210, the spectrophotometer 122 calculates the diffuse reflection signal, and the gingiva spectrum is generated. That is to say, for each gingiva to be evaluated, the spectrophotometer 122 may analyze the corresponding diffuse reflection signal to generate the corresponding gingiva spectrum.

In Step S220, the electronic device 110 applies the gingiva spectrum and a plurality of reference gingiva spectra respectively with a time-series similarity calculation, and a plurality of similarity values are generated. In this embodiment, the time-series similarity calculation includes the dynamic time warping (DTW) calculation. That is to say, the electronic device 110 compares the degree of similarity between the gingiva spectrum and each reference gingiva spectrum based on the DTW calculation. The degree of similarity may be expressed, for example, as a similarity value. In this embodiment, when the similarity value is large, it means that the distance (i.e., a distortion distance) between two spectral curves is large. Therefore, it means that the degree of similarity between the two spectral curves is low. On the contrary, when the similarity value is small, it means that the degree of similarity between the two spectral curves is high.

It should be noted that, the electronic device 110 calculates all wavelengths in the gingiva spectrum, instead of capturing and calculating part of the wave bands or a single wavelength in the gingiva spectrum.

In this embodiment, the reference gingiva spectra include a first reference gingiva spectrum to a fourth reference gingiva spectrum. The reference gingiva spectra correspond to different gingival indexes (GI). The gingival index may be distinguished from 0 to 3 to indicate the status of the gingiva as healthy, mildly inflamed, moderately inflamed, and severely inflamed. For example, the first reference gingiva spectrum (for example, a first reference gingiva spectrum SP_GIO shown in FIG. 3A) is the gingiva spectrum generated according to the gingiva with the gingival index of 0. The second reference gingiva spectrum is the gingiva spectrum generated according to the gingiva with the gingival index of 1, and so on. That is to say, in this embodiment, the similarity value corresponds to the reference gingiva spectrum that is most similar to the gingiva spectrum and corresponds to a specific gingival index.

In this embodiment, the reference gingiva spectrum may be, for example, a spectrum statistically calculated clinically, or a spectrum simulated based on a theoretical model. In an embodiment, the electronic device 110 may establish the multiple reference gingiva spectra based on the clinical gingiva spectrum and the baseline gingiva spectrum corresponding to a gingiva tissue. Specifically, the electronic device 110 applies the multiple clinical gingiva spectra and the baseline gingiva spectra respectively with calculations (for example, the DTW calculation) to generate multiple clinical similarity values. The clinical similarity value corresponds to the baseline gingiva spectrum that is most similar to the clinical gingiva spectrum and corresponds to a specific gingival index. In an embodiment, the electronic device 110 sets multiple similarity value threshold values according to the clinical similarity values and multiple clinical gingival indexes corresponding to the clinical gingiva spectra to generate the multiple reference gingiva spectra. For example, assuming that a first similarity value threshold value is set to 500. When the clinical similarity value is greater than 500, the corresponding clinical gingiva spectrum is output as a first reference gingiva spectrum. When the clinical similarity value is less than 500, the corresponding clinical gingiva spectrum is output as a second reference gingiva spectrum. In Step S230, the electronic device 110 obtains a minimum similarity value of the multiple similarity values. Since the lower the similarity value, the higher the degree of similarity between the gingiva spectrum and the reference gingiva spectrum, the electronic device 110 selects the minimum similarity value to generate the reference gingiva spectrum that is most similar to the gingiva spectrum.

In Step S240, the electronic device 110 outputs the gingival index result according to the minimum similarity value. Since each reference gingiva spectrum corresponds to a specific gingival index, the electronic device 110 generates the gingival index of the reference gingiva spectrum that is most similar to the gingiva spectrum according to the result of Step S230. The gingival index may be used as the content of a gingivitis diagnosis result (i.e., the gingival index result) to represent the health status of the gingiva.

It is worth mentioning that, the electronic device 110 performs the time-series similarity calculation (for example, the DTW calculation) according to the complete gingiva spectrum to analyze the degree of similarity between the gingiva spectrum and different reference gingiva spectra instead of capturing part of the wave bands or a single wavelength in the gingiva spectrum, thereby the accuracy of the evaluation is improved. On the other hand, the electronic device 110 generates the gingival index result according to the minimum similarity value of the DTW calculation, and can generate a gingivitis diagnosis result without the need for analysis or diagnosis by a doctor, thereby the efficiency of the evaluation is improved.

FIG. 3A to FIG. 3B are schematic operation diagrams of the gingivitis evaluating device shown in the embodiment of FIG. 1 according to the disclosure. Referring to FIG. 1 and FIG. 3A, the gingivitis evaluating device 100 may execute multiple Modules S310 to S370, in order to exemplarily illustrate the operation details of Step S220 shown in FIG. 2 regarding the time-series similarity calculation (for example, the DTW calculation) for the gingiva spectrum analysis, and Steps S230 to S240 shown in FIG. 2 regarding the operation details of generating the gingiva diagnosis result (for example, the gingival index result).

In Module S310, the electronic device 110 generates a gingiva spectrum SP1 of the gingiva to be evaluated output by the spectrophotometer 122. The electronic device 110 accesses the first reference gingiva spectrum SP_GIO in the memory. In this embodiment, the gingival index corresponding to the first reference gingiva spectrum SP_GIO is 0.

In Module S320, the electronic device 110 applies the gingiva spectrum SP1 of the first gingiva and the first reference gingiva spectrum SP_GIO with the DTW calculation. In this embodiment, even if the gingiva spectrum SP1 and the first reference gingiva spectrum SP_GIO have different time lengths or time series, by using the DTW calculation to distort the time axis of the spectra SP1 and SP_GIO, the spectra SP1 and SP_GIO may have approximately corresponding time series to calculate the distortion distances between respective time points.

In Module S330, the electronic device 110 generates the first similarity value corresponding to the first reference gingiva spectrum SP_GIO according to the calculation of Module S320. In this embodiment, the amount of the first similarity value is negatively correlated to the degree of similarity between the gingiva spectrum SP1 and the first reference gingiva spectrum SP_GIO.

In Module S340, the electronic device 110 replaces the first reference gingiva spectrum SP_GIO with a second reference gingiva spectrum (not shown), and repeatedly execute Modules S310 to S330 to generate a second similarity value corresponding to the second reference gingiva spectrum. In this embodiment, for the operations of the electronic device 110 generating a third similarity value and a fourth similarity value, reference may be made to the operations of Modules S310 to S330 and make analogies, so will not be repeated here. The third similarity value corresponds to the third reference gingiva spectrum, and the fourth similarity value corresponds to the fourth reference gingiva spectrum.

In Module S350, the electronic device 110 obtains the smallest of the similarity values (i.e., a minimum similarity value) according to the result of Module S340. The following embodiment illustrates by using the first similarity value as the minimum similarity value as an example.

In Module S360, the electronic device 110 outputs the gingival index corresponding to the first similarity value (for example, 0) as the gingival index result.

In Module S370, the electronic device 110 updates an oral examination record form of a patient according to the gingival index result and gingiva information of the patient. Specifically, the electronic device 110 accesses the gingiva information of the patient and the oral examination record form of the patient. The gingiva information may include a given number (or a tooth position) of the current gingiva to be evaluated. The oral examination record form may include basic information of the patient, multiple gingiva numbers and diagnosis fields thereof, and remarks fields. In this embodiment, the electronic device 110 fills the gingival index result (for example, the gingival index is 0) into the corresponding field in the oral examination record form (for example, the diagnostic field corresponding to the gingiva information). Therefore, the updated oral examination record form includes the gingival index result corresponding to the gingiva information.

In this embodiment, the electronic device 110 repeatedly executes Modules S310 to S370 until the evaluation of all gingivae are completed. In other words, multiple gingival index results have been filled in respective diagnostic fields in the last updated oral examination record form. It should be noted that, the gingivitis evaluating device 100 can automatically fill in the form to record the gingival index result of each gingiva information. It does not require the doctor to manually fill in the form every time when collecting the gingiva sensing data of a single gingiva, thereby the evaluating time is shortened and the operation efficiency is improved.

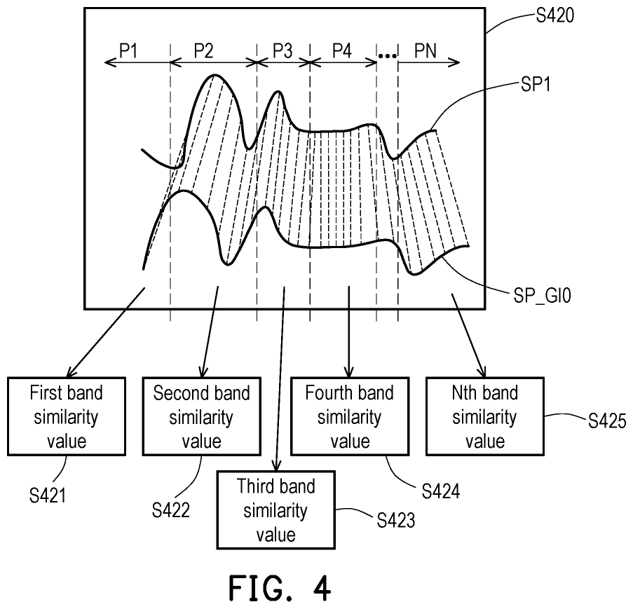

FIG. 4 is a schematic operation diagram of the gingivitis evaluating device shown in the embodiment of FIG. 1 according to the disclosure. Referring to FIG. 1 and FIG. 4, the gingivitis evaluating device 100 may execute multiple Modules S420 to S425, in order to exemplarily illustrate the operation details of Sep S220 shown in FIG. 2 regarding the time-series similarity calculation (for example, the DTW calculation) for the gingiva spectrum analysis. In this embodiment, Modules S420 to S425 may be, for example, another implementation of Module S320 shown in FIG. 3A.

In Module S420, the electronic device 110 applies the gingiva spectrum SP1 of the first gingiva and the first reference gingiva spectrum SP_GIO with the time-series similarity calculation. In this embodiment, the time-series similarity calculation includes the weighted dynamic time warping (WDTW) calculation. That is to say, the electronic device 110 uses the key spectral wave band weighted calculation method based on the DTW calculation.

In this embodiment, the electronic device 110 distinguishes the gingiva spectrum SP1 into multiple wave band gingiva spectra P1 to PN, in which N is a positive integer. The quantity and configuration of the wave band gingiva spectra P1 to PN in the embodiment of FIG. 4 are merely examples and are not limited thereto.

In this embodiment, taking N equal to 7 as an example, the wavelength of the wave band gingiva spectrum P1 is, for example, less than 400 nm. The wavelength of the wave band gingiva spectrum P2 is, for example, in a range of 400 nm to 500 nm. The wavelength of the wave band gingiva spectrum P3 is, for example, in a range of 500 nm to 550 nm. The wavelength of the wave band gingiva spectrum P4 is, for example, in a range of 550 nm to 580 nm. The wavelength of the wave band gingiva spectrum P5 is, for example, in a range from 580 nm to 620 nm. The wavelength of the wave band gingiva spectrum P6 is, for example, in a range from 620 nm to 700 nm. The wavelength of the wave band gingiva spectrum P7 is, for example, greater than 700 nm.

The various wave band gingiva spectra P1 to P7 may have different degrees of correlation with the gingivitis. For example, in the wave band gingiva spectrum P3, a specific wavelength (such as 545 nm or 575 nm) is correlated to hemoglobin (Hb) and oxygenated hemoglobin (HbO2). Hb and HbO2 are correlated to the degree of inflammation in gingiva. Therefore, compared to other wave bands, the wave band gingiva spectrum P3 is most correlated to the gingivitis.

In this embodiment, the electronic device 110 applies the respective wave band gingiva spectra P1 to PN and the first reference gingiva spectrum SP_GIO with the DTW calculation to generate multiple band similarity values. Specifically, in Module S421, the electronic device 110 applies the wave band gingiva spectrum P1 and the first reference gingiva spectrum SP_GIO with the DTW calculation to generate a first band similarity value. In Modules S422 to S425, for the operations of the electronic device 110 to generate a second band similarity value to an Nth band similarity value respectively, reference may be made to the relevant descriptions of Module S421 and make analogies, so will not be repeated here.

It should be noted that, since the degrees of correlation with the gingivitis among the wave band gingiva spectra P1 to P7 are different, the wave band gingiva spectra P1 to P7 may correspond to different weight values. That is to say, the weight values are correlated to gingivitis features (for example, the correlation between Hb and HbO2). At least two of the weight values are different from each other. The weight value may be, for example, a weight value of the distortion distance to enhance the difference in the degree of similarity between two spectral curves.

In this embodiment, the weight value is in a range of 0 to 1. When the weight value is large, it means that the distortion distance between the two spectral curves may be expanded. Therefore, it means that the degree of similarity between the two spectral curves is low. On the contrary, when the weight value is small, it means that the degree of similarity between the two spectral curves is high. For example, the wave band gingiva spectrum P3 correlated to the gingivitis correspond to a weight value of 0.5, and the wave band gingiva spectrum P4 correspond to a weight value of 0.6. The weight values corresponding to the remaining wave band gingiva spectra P1, P2, P5, P6, and P7 are 1, 0.9, 0.8, 0.9, and 1 respectively.

In this embodiment, the electronic device 110 multiplies the multiple weight values by the multiple band similarity values respectively to generate multiple weighted band similarity values. That is to say, the electronic device 110 weights the first band similarity value to the Nth band similarity value with weight values of different amounts respectively to obtain a first weighted band similarity value (i.e., the first weight value times the first band similarity value) to an Nth weighted band similarity value (i.e., the Nth weight value times the Nth band similarity value).

In this embodiment, the electronic device 110 sums the weighted band similarity values to generate the first similarity value corresponding to the first reference gingiva spectrum SP_GIO. In this embodiment, the electronic device 110 replaces the first reference gingiva spectrum SP_GIO with the second reference gingiva spectrum, and repeatedly executes Modules S420 to S425 to generate the second similarity value corresponding to the second reference gingiva spectrum. In this embodiment, for the operations of the electronic device 110 generating the third similarity value and the fourth similarity value, reference may be made to the operations of Modules S420 to S425 and make analogies, so will not be repeated here.

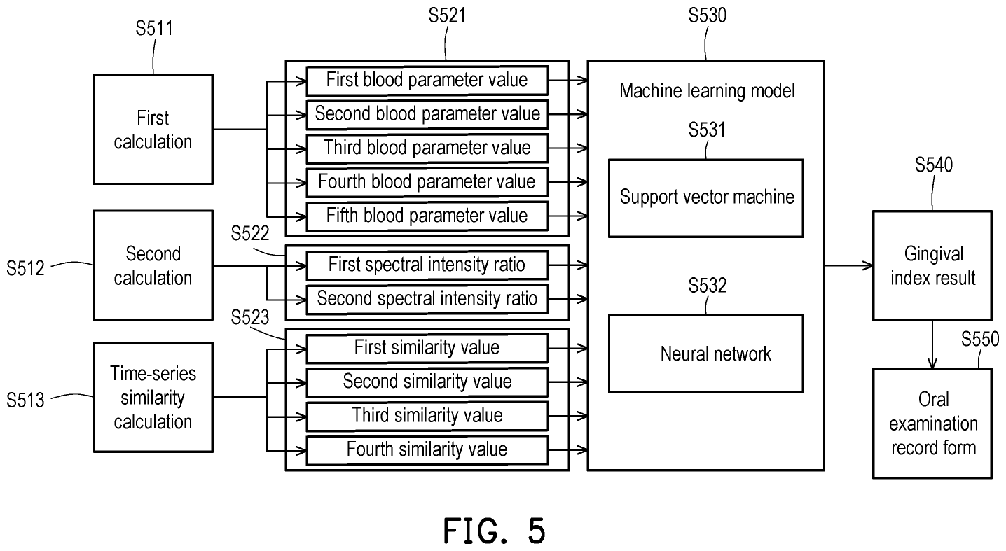

FIG. 5 is a schematic operation diagram of the gingivitis evaluating device shown in the embodiment of FIG. 1 according to the disclosure. Referring to FIG. 1 and FIG. 5, the gingivitis evaluating device 100 may execute multiple Modules S511 to S550 to evaluate the health status of the gingiva.

In Module S511, the electronic device 110 performs a first calculation according to the gingiva spectrum (for example, the gingiva spectrum SP1 shown in FIG. 3A). In this embodiment, the first calculation includes the electronic device 110 performing a chromophore fitting calculation on the gingiva spectrum to generate multiple blood parameter values. The blood parameter values may include multiple chromophore concentrations of multiple blood substances as physiological parameters for the gingiva. That is to say, the electronic device 110 performs spectral fitting on the gingiva spectrum with a variety of known chromophores to obtain the composition ratios of the chromophores.

In Module S521, the blood parameter values may exemplarily include a first blood parameter value to a fifth blood parameter value. For example, in this embodiment, the first blood parameter value is, for example, the chromophore concentration of N-Myeloperoxidase (N-MPO). The second blood parameter value is, for example, the chromophore concentration of C-myeloperoxidase (C-MPO). The third blood parameter value is, for example, the chromophore concentration of HbO2. The fourth blood parameter value is, for example, the chromophore concentration of Hb. The fifth blood parameter value is, for example, the sum of the third blood parameter value and the fourth blood parameter value.

In Module S512, the electronic device 110 performs a second calculation according to the gingiva spectrum (for example, the gingiva spectrum SP1 shown in FIG. 3A). In this embodiment, the second calculation includes the electronic device 110 analyzing the gingiva spectrum to generate a wavelength (for example, 620 nm) with the maximum peak in the gingiva spectrum. The second calculation also includes the electronic device 110 normalizing the gingiva spectrum with the wavelength to generate the multiple spectral intensity ratios. That is, the electronic device 110 divides the multiple wavelengths in the gingiva spectrum by the wavelength with the maximum peak to obtain a ratio between the corresponding amplitude and amplitude (i.e., the spectral intensity ratio).

In module S522, the spectral intensity ratio may exemplarily include a first spectral intensity ratio to a second spectral intensity ratio. For example, in this embodiment, the first spectral intensity ratio is, for example, the ratio between the amplitude corresponding to the wavelength 545 nm and the amplitude corresponding to the wavelength 620 nm. The second spectral intensity ratio is, for example, the ratio between the amplitude corresponding to the wavelength 575 nm and the amplitude corresponding to the wavelength 620 nm.

In Module S513 and Module S523, the electronic device 110 performs the time-series similarity calculation (for example, the DTW calculation) according to the gingiva spectrum (for example, the gingiva spectrum SP1 shown in FIG. 3A) to generate the first similarity value to the fourth similarity value. The operation details of Modules S513 and S523 may be found in the operations of Modules S310 to S340, so will not be repeated here.

In Modules S530 to S540, the electronic device 110 executes a trained machine learning model to generate the gingival index result according to at least one of the multiple blood parameter values, the multiple spectral intensity ratio values, and the multiple similarity values. That is to say, the electronic device 110 uses the result of the first calculation (i.e., the blood parameter value), the result of the second calculation (i.e., the spectral intensity ratio), and/or the result of the DTW calculation (i.e., the similarity value) as the input parameters of the machine learning model, and executes the machine learning model accordingly to output the gingival index result. In this embodiment, the machine learning model may include the support vector machine (SVM) and the neural network.

Specifically, in Module S531, the electronic device 110 executes the trained support vector machine. The trained support vector machine conducts inferences according to the multiple blood parameter values, the multiple spectral intensity ratios, and the multiple similarity values to generate inference results such as the gingival index, accuracy, sensitivity, and specificity. The electronic device 110 outputs the inference result as the gingival index result. In Module S532, the electronic device 110 executes the trained neural network. The trained neural network conducts inferences according to the multiple blood parameter values, the multiple spectral intensity ratios, and the multiple similarity values to generate the gingival index. The electronic device 110 outputs the gingival index as the gingival index result. In an embodiment, the trained neural network conducts inferences according to the inference result generated by Module S531 to optimize the accuracy of the gingival index and output the optimized gingival index as the gingival index result. In this embodiment, the neural network may be, for example, a model of a feed forward network or a recurrent network architecture.

It should be noted that, the electronic device 110 analyzes the gingiva spectrum and obtains three types of features such as the gingiva physiological parameter (i.e., the blood parameter value), the spectral intensity parameter (i.e., the spectral intensity ratio), and the spectral curve comparison parameter (i.e., the similarity value) accordingly. The trained support vector machine and/or neural network can infer the gingival index according to at least one of the three types of features to improve the accuracy of the evaluation. For example, a user may obtain the gingival index by selecting two types of features such as the gingiva physiological parameter and the similarity value through the user interface of the electronic device 110.

In Module S550, the electronic device 110 updates the oral examination record form of the patient according to the gingival index result and the gingiva information of the patient. The operation details of Module S550 may be found in the operation of Module S370, so will not be repeated here.

In summary, in the spectrum analyzing method and the gingivitis evaluating device according to the embodiments of the disclosure, the DTW calculation is performed according to the gingiva spectrum to determine the most similar spectrum between the gingiva spectrum and the spectrum corresponding to various gingival indexes (i.e., the reference gingiva spectrum), instead of analyzing part of the wave bands or the wavelength, and the accuracy of the evaluation can be improved. In addition, the gingivitis evaluating device generates the gingivitis diagnosis result (i.e., the gingival index result) according to the result of the DTW calculation, which can improve the efficiency of the evaluation. In some embodiments, the gingivitis evaluating device automatically completes the form filling operation of the oral examination record form according to the gingival index result, which can shorten the evaluation time and improve the efficiency of the evaluation.

Although the disclosure has been disclosed above in the embodiments, the embodiments are not intended to limit the disclosure. Persons with ordinary knowledge in the technical field may make some changes and modifications without departing from the spirit and scope of the disclosure. Therefore, the protection scope of the disclosure shall be determined by the appended claims.

What is claimed is:

1. A spectrum analyzing method, comprising:
calculating a diffuse reflection signal of a gingiva and generating a gingiva spectrum;
applying the gingiva spectrum and a plurality of reference gingiva spectra respectively with a time-series similarity calculation and generating a plurality of similarity values, wherein the plurality of reference gingiva spectra correspond to various gingival indexes;
obtaining a minimum similarity value of the plurality of similarity values; and
outputting a gingival index result according to the minimum similarity value.

2. The spectrum analyzing method as claimed in claim 1, wherein the time-series similarity calculation comprises a dynamic time warping (DTW) calculation.

3. The spectrum analyzing method as claimed in claim 1, further comprising:
updating an oral examination record form of a patient according to the gingival index result and gingiva information of the patient, so that the oral examination record form comprises the gingival index result corresponding to the gingiva information.

4. The spectrum analyzing method as claimed in claim 1, wherein applying the gingiva spectrum and the plurality of reference gingiva spectra respectively with the time-series similarity calculation and generating the plurality of similarity values comprises:
distinguishing the gingiva spectrum into a plurality of wave band gingiva spectra;
applying each of the plurality of wave band gingiva spectra and each of the plurality of reference gingiva spectra with the time-series similarity calculation to generate a plurality of band similarity values;
multiplying a plurality of weight values by the plurality of band similarity values respectively to generate a plurality of weighted band similarity values; and
summing the plurality of weighted band similarity values to generate one of the plurality of similarity values.

5. The spectrum analyzing method as claimed in claim 4, wherein at least two of the plurality of weight values are different, and the plurality of weight values are correlated to gingivitis features.

6. The spectrum analyzing method as claimed in claim 1, further comprising:
performing a first calculation according to the gingiva spectrum to generate a plurality of blood parameter values;
performing a second calculation according to the gingiva spectrum to generate a plurality of spectral intensity ratios; and
executing a trained machine learning model to generate the gingival index result according to at least one of the plurality of blood parameter values, the plurality of spectral intensity ratios, and the plurality of similarity values.

7. The spectrum analyzing method as claimed in claim 6, wherein performing the first calculation according to the gingiva spectrum to generate the plurality of blood parameter values comprises:
performing a chromophore fitting calculation on the gingiva spectrum to generate the plurality of blood parameter values, wherein the plurality of blood parameter values comprise a plurality of chromophore concentrations of a plurality of blood substances.

8. The spectrum analyzing method as claimed in claim 6, wherein performing the second calculation according to the gingiva spectrum to generate the plurality of spectral intensity ratios comprises:
generating a wavelength with a maximum peak in the gingiva spectrum; and
normalizing the gingiva spectrum with the wavelength to generate the plurality of spectral intensity ratios.

9. The spectrum analyzing method as claimed in claim 1, further comprising:
applying a plurality of clinical gingiva spectra and baseline gingiva spectra respectively with the time-series similarity calculation to generate a plurality of clinical similarity values; and
setting a plurality of similarity value threshold values according to the plurality of clinical similarity values and a plurality of clinical gingival indexes corresponding to the plurality of clinical gingiva spectra to generate the plurality of reference gingiva spectra.

10. A gingivitis evaluating device, comprising:
a sensor configured to sense a diffuse reflection signal of a gingiva;
a spectrophotometer coupled to the sensor and configured to calculate the diffuse reflection signal to generate a gingiva spectrum; and
an electronic device coupled to the spectrophotometer and configured to:
apply the gingiva spectrum and a plurality of reference gingiva spectra respectively with a time-series similarity calculation and generate a plurality of similarity values, wherein the plurality of reference gingiva spectra correspond to various gingival indexes;
obtain a minimum similarity value of the plurality of similarity values; and
output a gingival index result according to the minimum similarity value.
